(19) United States
(12) Patent Application Publication
(10) Pub. No.: US 2009/0132023 A1
(43) Pub. Date: May 21, 2009

(54) METHODS AND APPARATUS WITH POROUS MATERIALS

(76) Inventors: Karl Sieradzki, Scottsdale, AZ (US); Jack Lundeen, Scottsdale, AZ (US)

Correspondence Address: THE NOBLITT GROUP, PLLC, 4800 NORTH SCOTTSDALE ROAD, SUITE 6000, SCOTTSDALE, AZ, 85251, US

(21) Appl. No.: 12/195,349

(22) Filed: Aug. 20, 2008

Related U.S. Application Data
(63) Continuation of application No. 10/840,200, filed on May 5, 2004, now abandoned.

(51) Int. Cl. A61F 2/06

(52) U.S. Cl. 623/1.15; 623/1.44; 623/1.46; 623/1.39; 623/1.42

(57) ABSTRACT

A method and apparatus according to various aspects of the present invention comprises a system having multiple pores. In one embodiment, the system comprises a medical device for insertion into an organism, comprising a main structure and a porous portion on the main structure.

### RELATED APPLICATIONS

This application is a continuation of and claims priority to U.S. patent application Ser. No. 10/840,200, filed May 5, 2004 by Karl Sieradzki and Jack Lundeen in the United States Patent and Trademark Office.

### BACKGROUND OF THE INVENTION

1. Field of the Invention

The present invention pertains generally to methods and apparatus relating to porous materials.

2. Description of Related Art

Porous materials, particularly porous metals, find uses in a variety of applications, such as catalytic converters, membranes, filters, sensors, and batteries. Several processes for making and utilizing porous metals have been developed and deployed. Such techniques, however, may benefit from greater simplicity and faster production. In addition, the manufacture and use of porous materials may gain from techniques that provide greater reproducibility and reliability, such as to control pore size, density, and distribution. Furthers porous material processes typically profit by using less expensive and safer materials.

Medical devices, on the other hand, are also widely used in a variety of applications, such as for placement in a lumen of a patient. For example, intraluminal devices such as stents are commonly used to treat obstructed coronary arteries. Stents are typically placed on a balloon tip catheter or sheath and advanced through the patient's blood vessels to an occluded artery. At the occluded site, the stent is expanded to enlarge its diameter. With the stent so enlarged, the catheter or sheath is removed from the patient, leaving the enlarged stent in place with the intent that the formerly occluded site is held open by the stent.

In addition to advancing and deploying stents as described above, catheters are used in a wide variety of applications within the body. Other medical devices, such as pacemakers, prosthetics, surgical tools, bone screws and anchors, sutures, and plates may also be placed in the body of a patient, either temporarily or permanently. Introduction of foreign objects into the body, however, may have adverse effects, such as scarring, rejection, and other problems. The caregiver often responds to the adverse effects with drugs or other chemicals to counter the symptoms or causes. The drugs or other chemicals are ordinarily delivered via conventional mechanisms, such as intravenous administration.

### BRIEF SUMMARY OF THE INVENTION

Methods and apparatus according to various aspects of the present invention comprise a system having multiple pores. In one embodiment, the system comprises a medical device for insertion into an organism, comprising a main structure and a porous portion on the main structure.

### BRIEF DESCRIPTION OF THE DRAWING

The invention will be more fully understood by reference to the following drawings which are for illustrative purposes only:

FIGS. 1A-C are cross-sectional views of medical devices according to various aspects of the present invention.

FIG. 2 is a perspective view of a stent structure.

FIG. 3 is flow chart of a method for forming pores.

FIG. 4 is cross-section view of a medical device being processed to form pores in a surface.

FIG. 5 is cross-section view of a medical device having an adhesion layer being processed to form pores in a surface.

FIG. 6 is a table illustrating the resulting pore sizes for various materials and annealing temperatures.

### DETAILED DESCRIPTION OF EXEMPLARY EMBODIMENTS

The present invention is described partly in terms of functional components and various processing steps. Such functional components may be realized by any number of components configured to perform the specified functions and achieve the various results.

For example, the present invention may employ various elements, materials, surfaces, adhesion layers and materials, medical device materials, porous materials, solvents, and the like, which may carry out a variety of functions. In addition, the present invention may be practiced in conjunction with any number of applications, environments, porous structures, and surfaces, and the systems described are merely exemplary applications for the invention. Further, the present invention may employ any number of conventional techniques for manufacturing, preparation, deployment, and the like.

A method and apparatus according to various aspects of the present invention comprises a system having pores. The porous system may be used for any suitable purpose or combination of purposes, such as to filter or separate gases, liquids, impurities or targeted substances; affect the structural strength of the apparatus; deliver substances to an environment through impregnating the pores with the substance to be delivered and placing the apparatus in an environment capable of removing or consuming the substance from the pores; capturing substances wherein the apparatus with porous material is placed in an environment where the substance to be removed passes from the environment into the pores; transferring electric potential; sensing electromagnetic fields; or any other suitable application. The method and apparatus may be adapted for any system using a porous portion for any purpose.

For example, porous systems according to various aspects of the present invention wholly or partially comprised of material with pores may be configured as medical devices of various shapes, sizes, and function for use internally or externally to the body on a permanent or temporary basis. Suitable medical devices with pores may include catheters, stents, bone anchors, surgical wires, or other devices. The pores may be configured in accordance with any desired function, such as filtration, drug delivery, or promotion of integration with surrounding tissue.

In one embodiment, a microporous system according to various aspects of the present invention comprises a medical device including a stent, such as a stent for implantation in a blood vessel or other bodily duct. The stent includes a portion having pores, such as a metal portion having pores formed in it. The porous portion may be used for any suitable purpose, such as for the delivery of drugs following implantation.

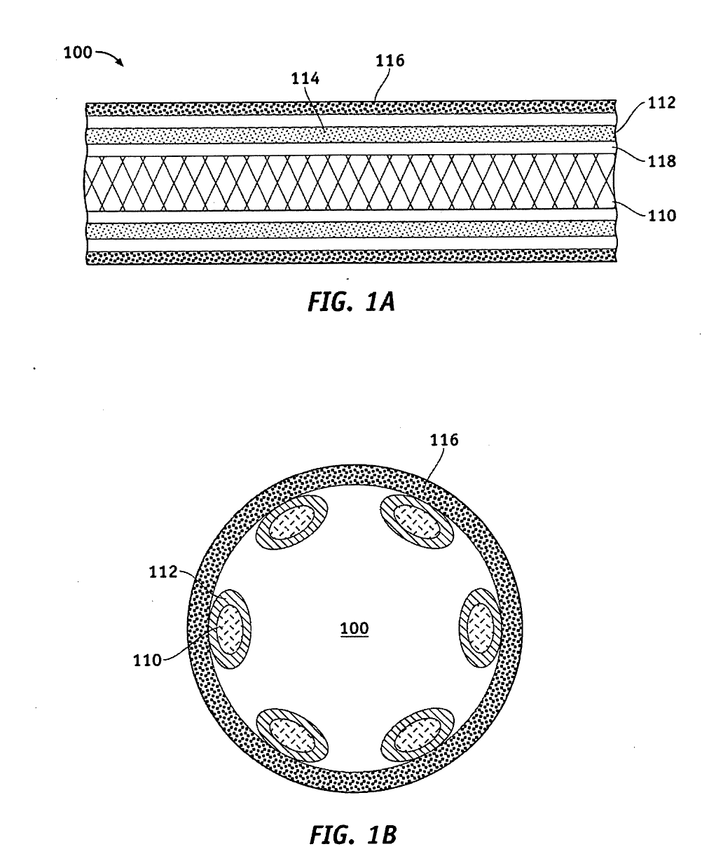

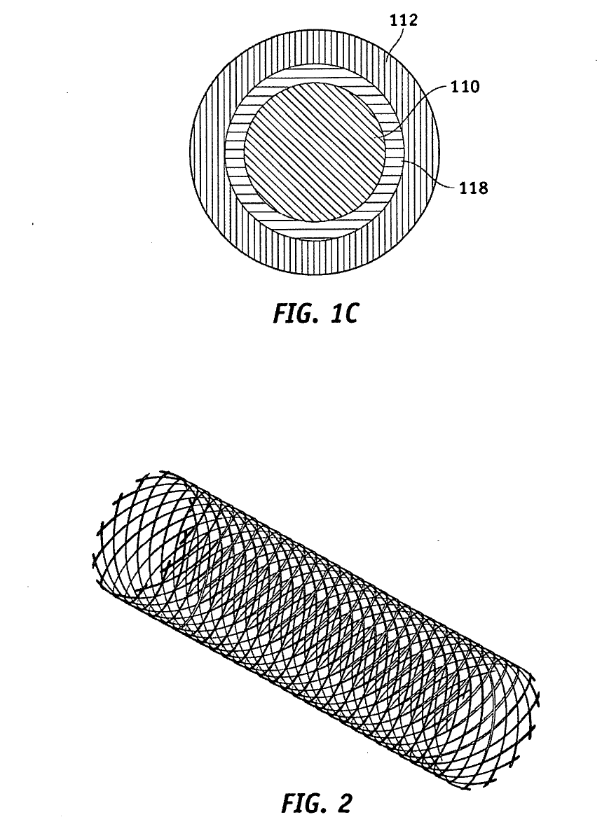

In particular, referring to FIGS. 1A-B, a stent 100 according to various aspects of the present invention comprises a stent structure 110, a porous portion 112, an elution material 114, and a package 116. The stent 100 is suitably a vascular stent for maintaining an opening in a blood vessel and/or relieving pressure on a blood vessel wall. The stent structure 110 provides the main structural support for the stent 100 to maintain the integrity of the stent 100. The porous portion 112 comprises a material having multiple small voids or pores formed therein. The porous portion 112 may serve any suitable purpose, such as retaining the elution material 114 for delivery following implantation. The package 116 provides a covering over at least a portion of the stent structure 110, the porous portion 112, and the elution material 114, for example to preserve the drug within the porous portion 112 and/or maintain the sterility of the various elements within the package 116.

More particularly, the stent structure 110 comprises any suitable structure for implantation in the particular application. Referring to FIG. 2, in the present embodiment, the stent structure 110 comprises a conventional stent configured for delivery into a vessel and expansion within the vessel. The stent structure 110 may be configured in any suitable manner, for example as a balloon-expandable stent or a self-expanding stent. The stent structure 110 of the present embodiment comprises a conventional stent configuration to facilitate delivery of the stent 100 and retention of the stent 100 in position. The stent structure 110 may also be comprised of any appropriate material, such as a biocompatible material that substantially retains its shape following deployment. In the present embodiment, the stent structure 110 may comprise stainless steel, gold, platinum, an alloy, a polymer, a composite, or other suitable material.

The porous portion 112 of the present stent 100 is disposed on the stent structure 110, for example attached to or integrated into the stent structure 110. The porous portion 112 may comprise any portion of the stent 100, such as an exterior or interior portion, an end portion, or the entirety of the stent 100 such that the porous portion 112 comprises the entire stent structure 110.

The porous portion 112 comprises a portion of the stent 100 having small voids or pores formed in it, typically having diameters of less than about one millimeter, and in the present embodiment, less than about ten micrometers, although the pores may be formed over any suitable range of sizes, such as 5-1000 nm. The pores may be situated in any part of the stent structure 110, such as on the surface of or embedded in the stent structure 110. The distribution of the pores according to size and/or density in or on the stent structure 110 may be uniform, non-uniform, or distributed according to a gradient or other scheme. Further, the size of the pores may be of equal, unequal, or random size. The porous portion may comprise any suitable material, such as metal, metal alloy, semi-metal, plastic, glass, ceramic, polymers, composite materials, or any other material capable of supporting the relevant pores.

The porous portion 112 may be formed on the surface of the stent structure 110, embedded into the stent structure 110, or integrated into the stent structure 110. Referring again to FIG. 1A-B, in the present exemplary embodiment, the porous portion 112 comprises a surface portion of the stent structure 110. The surface may be an exterior surface, an interior surface, or any other suitable surface of the stent structure 110, and may cover all or only a portion of the relevant surface. The porous structure 112 may also have any suitable depth on the stent structure 110. In the present embodiment, for a vascular stent having a diameter of, for example, five millimeters, the porous portion 112 has a depth of approximately 1,000 to 10,000 Angstroms.

The porous portion 112 is suitably comprised of a biocompatible material, such as gold, platinum, titanium, or stainless steel. The stent structure 110 and the porous portion 112 may be comprised of the same or different materials. For example, the stent structure 110 may comprise a stainless steel, and the porous portion 112 may comprise stainless steel, gold, titanium, platinum, or other suitable material.

The porous structure 112 of the present embodiment has pores defined within the structure to retain the elution material 114, such as a fluid drug for elution by the body following implantation. The pores may comprise any suitable structure, density, and/or size for retaining the relevant drug. In the present embodiment, the pores may be any appropriate size, such as from approximately 1 nm to about 10,000 nm, for example about 5-1000 nm. The size and/or density of the pores may vary, such as along the length of the stent structure 110, to facilitate extended release of the eluted drug.

Referring to FIG. 1C, the stent 100 may also include an adhesion material 118 to assist in retaining the porous portion 112 in position with respect to the stent structure 110. For example, if the stent structure 110 and the porous structure 112 comprise certain different materials, the porous portion 112 material may tend to separate or peel away from the stent structure 110. The adhesion layer 118 suitably comprises an interface material that bonds to both the stent structure 110 and the porous structure 112. For example, if the stent structure 110 comprises stainless steel and the porous portion 112 comprises gold, a suitable adhesion layer 118 may comprise chromium disposed between the stent structure 110 and the porous structure 112. The adhesion layer may comprise any suitable thickness, such as about 25-75 Å, for example about 50 Å.

Referring again to FIGS. 1A-B, the pores of the porous portion 112 may remain open, for example to impart desired structural characteristics or weight characteristics or to absorb or filter fluids. Alternatively, the porous portion 112 may be impregnated, partially or wholly, with a compound or substance, such as the elution material 114 for transfer from the pores into the body following implantation. The elution material 114 may comprise any suitable drug, such as an anti-scarring agent, an anti-inflammatory agent, or an anti-biotic.

The package 116 is suitably configured to at least partially enclose the stent 100. The package 116 may be configured for any suitable purpose, such as to prevent contamination of the stent, preserve the elution material 114, or prevent damage to the stent 100. In the present embodiment, the package 116 completely envelops the other components of the stent 100. The package 116 may comprise any suitable material, such as a plastic or cloth. The package 116 is suitably removed before the stent 100 is implanted in the body. If deemed unnecessary, however, the package 116 may be omitted from the stent 100.

The porous system may be prepared according to any suitable process for the particular application and/or environment. Small medical devices, such as stents, catheters, surgical screws and anchors, and the like, may be prepared using biocompatible materials and under conditions for maintaining precision and purity. Other applications, however, may require different standards, materials, and procedures for forming the porous system.

In the present embodiment, the stent 100 having the porous portion 112 may be formed according to any suitable process. In accordance with one embodiment, the stent 100 is formed by creating a material having a porous portion 112 and forming the stent 100 from the material having the porous portion 112. In another embodiment, the stent 100 is formed by generating the stent 100 and then forming the porous portion on or in the stent. Further, the porous portion 112 may be provided by forming pores in the stent structure 110 itself or in a different pore formation material. The pores may be formed in any suitable manner, such as by exposing the stent structure 110 or the pore formation material to a solvent for leaching out components of the stent structure 110 or the pore formation material, leaving the porous portion 112 as a result.

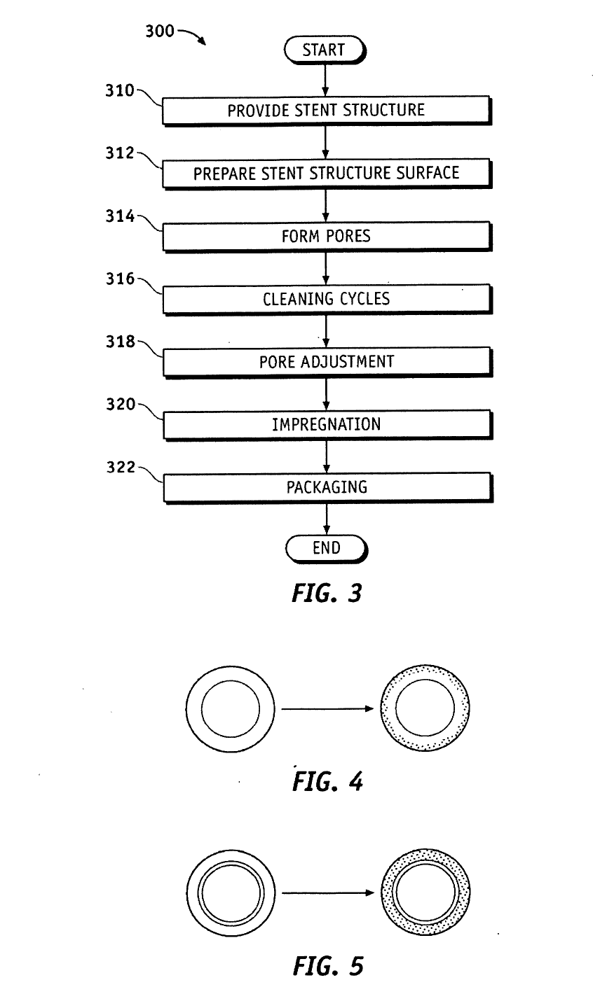

For example, referring to FIG. 3, an exemplary process 300 for forming the stent 100 according to various aspects of the present invention comprises providing the stent structure 110 of the desired stent structure material (310). The stent structure 110 may be generated according to any appropriate process or technique, such as via conventional stent manufacturing.

The porous portion 112 may be generated or deposited in any suitable manner according to any appropriate criteria, such as the materials to be used, the environment and/or application for the stent 100 or other porous system, cost concerns, and the like. In the present embodiment, the porous portion 112 is formed on and/or in the stent structure 110. To form the porous portion 112, the relevant surface of the stent structure 110 may be prepared (312). For example, the relevant surface may be cleaned of contaminants. Additional preparation may include, among other things, heating, shaping, stressing, laser scoring, mounting, or other processes to prepare the stent structure 110 before formation or placement of the porous portion 112.

The preparation of the stent structure 110 surface may also be adjusted according to the process for forming or depositing the porous portion 112. For example, if the porous portion 112 is to be formed directly into the surface of the stent structure 110, such as a stent structure 110 comprising an alloy, no additional preparation may be required, A stainless steel stent, for example, may be treated to remove iron, chromium, or nickel to form the porous portion 112.

Alternatively, to facilitate formation of the pores, the relevant surface of the stent structure 110 may be exposed to impurities, such as using conventional diffusion techniques to dope the relevant portion of the stent structure 110 with selected materials. For example, a gold stent structure 110 may be diffused with silver. The concentration of silver diffused into the gold stent may be selected according to the desired size and/or density of the pores.

Alternatively, additional pore formation materials may be deposited on the stent structure 110 to facilitate formation of the porous structure 112. The pore formation material may be deposited according to any suitable process and/or technique, such as vapor deposition, sputtering, thin-film deposition, electro-chemical deposition, or any other appropriate method. The pore formation materials may be deposited to any desired thickness. For example, in the present stent 100 embodiment, the pore formation material comprises a substantially even layer of material of substantially the same thickness as the desired final thickness of the porous portion 112.

If the pore formation material does not sufficiently adhere to the stent structure 110, the stent structure 110 and/or the pore formation material may be treated to promote adhesion. For example, the surface of the stent structure 110 may be scored to promote a mechanical bond. In the present embodiment, the adhesive layer 118 may be deposited, inserted, injected, or otherwise attached to or associated with the relevant surface of the stent structure 110.

To form the porous portion 112, pores are formed in the stent structure 110 and/or the pore formation material (314). The pores may be formed using any suitable process for forming pores of a desired size, consistency, distribution, and/or density. For example, the pores may be formed using micro-machining, micro-boring, nano-technology, material deposition, electrolytic processes, chemical etching, plasma etching, photoresistive processes combined with etching, or leaching. In addition, the pore formation process may be configured according to the process used to form the pores, the materials involved, and/or any other suitable considerations. For example, the pore formation process may include variations in process temperature and pressure, an inert atmosphere, agitation, spinning, vibrating, sonic exposure, or any other environmental or physical alteration.

In the present embodiment, the pores are formed in conjunction with a leaching process. The leaching process may be performed in any suitable manner, such as a free corrosion etch or in conjunction with the application of a voltage. The leaching process exposes the relevant portion of the stent structure 110 and/or the pore formation material to a solvent that is configured to dissolve one or more target components in the stent structure 110 and/or the pore formation material without materially affecting other components. When the target components are leached from the stent structure 110 and/or the pore formation material, the remaining material forms the porous portion 112.

For example, referring to FIG. 4, the stent structure 110 is suitably comprised of stainless steel. To form the pores, the stent is placed in a solvent configured to dissolve at least one component of the stainless steel without significantly affecting another component of the stainless steel. For example, the stainless steel stent structure 110 is suitably placed in a 50% solution of sodium hydroxide at approximately 140° C. The solution leaches iron from the stainless steel, leaving pores. The solution can be altered or different solutions applied, serially or simultaneously, to leach different materials from the stainless steel, such as chromium, nickel, or other materials present in the stainless steel. In addition, the temperature of the leaching process may be adjusted to control the outcome. For example, in the present embodiment, the temperature of the sodium hydroxide may be reduced to during the leaching process, for example to 10° C., to reduce the size of the pores.

Similarly, referring to FIG. 5, a gold-silver alloy deposited on an adhesion layer atop a stainless steel stent structure may be exposed to a solvent to leach out either the silver or the gold. The alloy may include any suitable alloy, such as a gold-silver alloy having about 20-50% (by atomic percentage) gold, for example approximately 22-30% gold. Because gold is substantially biocompatible, the silver is suitably leached in the present embodiment by exposing the silver-gold alloy to a nitric acid solution for between 10 and 30 minutes at standard temperature and pressure. The acid solution may be any suitable solvent, such as an approximately 40-60% nitric acid solution, for example a 50% nitric acid solution. The nitric acid solution leaches silver from the gold-silver alloy, leaving porous gold. In an alternative embodiment using a platinum-copper alloy, a similar solution of nitric acid may be used to leach copper from the platinum-copper alloy. Further, the platinum-copper alloy may comprise any suitable alloy, such as a platinum-copper alloy having about 20-50% (by atomic percentage) platinum, for example approximately 22-30% platinum.

The size, location and density of pores may be selected according to the method used to form the pores. If the process of forming pores is subtractive, removing more material results in large pores. If the process is additive, adding more material results in smaller pores. Leaching selectively removes the materials that react with the leaching solution. A higher concentration of the reactive material in the stent structure 110 and/or the pore formation material provides a greater the amount of material potentially removed, thereby forming larger pores. In addition, the pore size may be controlled by one or more variables. For example, the pore size may be controlled by adjusting the composition of the materials, applying heat treatment, adjusting the speed of the process such as by applying a voltage, or adjusting the concentration of the solvent.

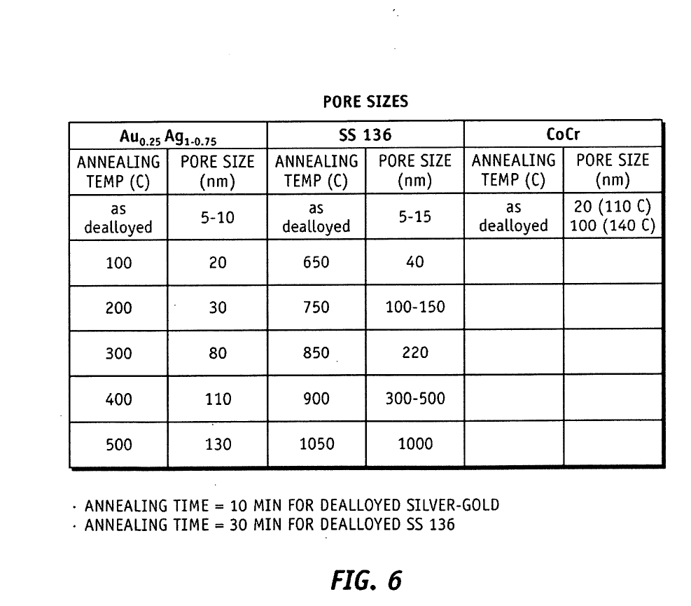

Referring now to FIG. 6, the duration and efficiency of the leaching process also affects the amount of material removed and the size of the remaining pores. In the first embodiment, iron is the most prevalent material in stainless steel, so leaching iron can result in a higher density of pores than leaching another material from the alloy. In particular, leaching iron from an SS316 stainless steel results in a pore size of about 5-15 nm. Similarly, for a silver-gold alloy, increasing the concentration of silver in the gold-silver alloy increases the amount of material potentially removed and thereby the pore density. A 75% concentration (by atomic percentage) of silver in the gold-silver alloy results in approximately 5-10 nm pores of relative even distribution. Likewise, the concentration of copper in the platinum-copper alloy affects the maximum amount of material potentially removed and the pore-size. Increasing the copper concentration increases the maximum potential pore density. A 25% concentration of copper in platinum results in a pore of approximately 3 nm after leaching.

Location and density of pores may be controlled by the spatial distribution of the material that reacts with the leaching solution. Material preparation techniques may be adapted to distribute reactive material according to the desired resulting pore pattern, location or density. Uniform distribution of the reactive material, and hence the resulting pores, is not mandatory.

The stent 100 is suitably cleaned to remove unwanted materials (316). For example, after the leaching process to form the pores in the present embodiment, the stent 100 is suitably rinsed in a neutral liquid, such as water, to remove solvent and leached materials. For example, the stent 100 may be placed in water for a period of time, such as ten minutes. The process of rinsing the stent 100 may be repeated as needed. The stent 100 may also be dried, for example in an inert atmosphere to avoid oxidation or other degradation of the stent 100 materials.

The porous portion 112 may also be treated to adjust the size of the pores to achieve a desired size and/or configuration of the pores (318). For example, all or a portion of the porous portion 112 may be treated to enlarge or coarsen the treated pores. Any suitable process may be applied to the porous portion 112 to refine the characteristics of the porous portion 112.

For example, in the present embodiment, the porous portion 112 is suitably exposed to heat to adjust the pore size by annealing. Annealing causes a clumping or coarsening of the pore structure, resulting in larger pores. Increasing the annealing time and/or temperature increases the pore size. The annealing process may be performed under any appropriate conditions. For example, referring again to FIG. 6, a gold porous portion 112 starting with about 5-10 nm pore diameter is suitably exposed to a temperature between about 200° C. and 500° C. for about ten minutes, to arrive at a final pore diameter of between about 20 and 130 nm. The temperature and time of exposure may be adjusted to control the amount of coarsening and the resulting pore size. If the materials to be exposed are subject to oxidation or other unwanted effects, the annealing process may be performed in a substantial vacuum or inert atmosphere. Similar results may be obtained using other materials, such as SS 316 stainless steel.

If the elution material 114 is to be included, the stent 100 is suitably impregnated with the desired material (320). The pores of the stent 100 may be impregnated by any method including, but not limited to, dipping, spraying, or depositing. The elution material 114 selected may adhere to the surfaces defining the pores. Some drugs may adhere more effectively to various materials than others. Consequently, the material of the porous portion 112 may be selected, at least in part, according to the characteristics of the elution material 114.

The package 116 may be applied to the stent 100 in any suitable manner (322). The package 116 may serve one or more purposes, such as protecting the apparatus before use, marketing, identification, preservation of the compound impregnated in the pores, and sterilization. The package 116 is suitably removed from the stent 100 before use.

Although the description above contains many details, these should not be construed as limiting the scope of the invention but as merely providing illustrations of some of the embodiments of this invention. The scope of the present invention fully encompasses other embodiments, and the scope of the present invention is accordingly limited by nothing other than the appended claims, in which reference to an element in the singular is not intended to mean “one and only one” unless explicitly so stated, but rather “one or more.” All structural, chemical, and functional equivalents to the elements of the above-described preferred embodiment are expressly incorporated by reference. Furthermore, no element, component, or method step in the present disclosure is intended to be dedicated to the public regardless of whether the element, component, or method step is explicitly recited in the claims.

1. A medical device for insertion into an organism, comprising:
a main structure; and
a substantially rigid porous portion on the main structure.

2. A medical device according to claim 1, wherein the main structure comprises a stent.

3. A medical device according to claim 1, wherein the porous portion includes a porous metal.

4. A medical device according to claim 1, wherein the porous portion is at least one of attached to or integrated into the main structure.

5. A medical device according to claim 1, wherein the porous portion is attached to the main structure by an adhesion layer.

6. A medical device according to claim 1, further comprising an elution material, wherein the elution material is at least partially retained within the porous portion.

7. A medical device according to claim 6, wherein the elution material is a drug.

8. A medical device according to claim 1, wherein the porous portion has defined therein pores having different sizes.

9. A medical device according to claim 1, wherein:
the main structure includes stainless steel; and
the porous portion includes gold.

10. A medical device according to claim 9, further comprising an adhesion layer including chromium between the main structure and the porous portion.

11. A stent according to claim 1, wherein the stent comprises:
a stent structure; and
a porous portion on the stent structure, wherein the surface comprises a surface of the porous portion.

12. A stent according to claim 11, wherein the porous portion is at least one of attached to or integrated into the stent structure.

13. A stent, comprising:
a stent structure; and
a porous portion on the stent structure, wherein the porous portion includes a porous metal.

14. A stent according to claim 13, wherein porous portion is at least one of attached to or integrated into the stent structure.

15. A stent according to claim 13, wherein the porous portion is attached to the stent structure by an adhesion layer.

16. A stent according to claim 13, further comprising an elution material, wherein the elution material is at least partially retained within the porous portion.

17. A stent according to claim 16, wherein the elution material is a drug.

18. A stent according to claim 13, wherein the porous portion has defined therein pores having different sizes.

19. A stent according to claim 13, wherein:
the stent structure includes stainless steel; and
the porous portion includes gold.

20. A stent according to claim 19, further comprising an adhesion layer including chromium between the main structure and the porous portion.
